(19) United States
(12) Patent Application Publication
(10) Pub. No.: US 2018/0264067 A1
(43) Pub. Date: Sep. 20, 2018

(54) Feed And Feed Supplement Formulation

(71) Applicants: Sandra M. Calveley, Duncan (CA); Leslie L. Kimmett, Sooke, CA (US)

(72) Inventors: Sandra M. Calveley, Duncan (CA); Leslie L. Kimmett, Sooke, CA (US)

(21) Appl. No.: 15/918,248

(22) Filed: Mar. 12, 2018

Related U.S. Application Data
(60) Provisional application No. 62/473,032, filed on Mar. 17, 2017.

(51) Int. Cl. A61K 36/899; A61K 36/185; A61K 31/352; A61K 36/31; A61K 36/23; A61K 33/26; A61K 9/00; A61P 1/00; A23K 10/30; A23K 20/28; A23K 50/40; A23K 50/20

(52) U.S. Cl. CPC A61K 36/899; A61K 36/185; A61K 31/352; A61K 36/31; A61K 36/23; A23K 50/20; A61K 9/0056; A61P 1/00; A23K 10/30; A23K 20/28; A23K 50/40; A61K 33/26

(57) ABSTRACT

A formulation for alleviating digestive tract issues in animals, the formulation including oat flour, hemp flour, dehydrated cabbage, dehydrated carrot and optionally diatomaceous earth.

### FIELD OF THE INVENTION

The present invention relates to feed and feed supplement formulations aimed at alleviating digestive tract issues in animals.

### BACKGROUND OF THE INVENTION

Horses and dogs frequently suffer from digestive tract issues.

Research indicates that horses have a rate of gastric and stomach ulcers of between 65% and 90%. Additionally, 4-5% of horses have colic every year, with 6.4% of those cases resulting in death. Horses with these and other digestive tract issues may have secondary health or pain issues. These can display as negative behavioral issues, unwillingness to work, lameness issues, poor coat and hoof health, muscular issues, etc.

It is not uncommon for high performance horses to be in pain and constantly under vet care, for various gastrointestinal ailments, including ulcers, colic, diarrhea and leaky gut syndrome (damaged tract lining enabling bacteria, toxins and large molecules to pass into the bloodstream leading to toxicity, laminitis or founder). Some research studies indicate that 80% of horses have gastric ulcers, 60% have colonic ulcers and that 50% have ulcers in both stomach and colon. Colic is a leading cause of death in horses. Chronic diarrhea can lead to dehydration and kidney failure.

Known treatments for ulcers in horses involve the use of pharmaceutical products, being proton pump inhibitors, which stop the production of stomach acids, thereby allowing the ulcers to heal. Horses treated for ulcers often have recurring issues and require repeated treatments. The cost of treatment through pharmaceuticals may be $2,500 per month. Treatment is often required more than once a year.

Known treatments for mild colic in horses include use of analgesics or sedatives to relieve pain and distress, antispasmodics, laxatives to help re-establish normal intestinal function, IV hydration, and passage of stomach tube to determine presence of excess gas, fluid or ingesta. Severe colic may require emergency surgery at a cost of $4000-$8000.

Known treatments for chronic diarrhea in horses are fluids and electrolytes to offset dehydration, kaolin and pectin, anti-inflammatory drugs and plasma to ward off endotoxemia, sepsis and its associated bacteria.

Research indicates that dogs have a high incidence of gastric and stomach issues including acidosis, colon distension, leaky gut syndrome (caused by damage to the digestive tract lining enabling bacteria, toxins and large molecules to pass into the bloodstream leading to toxicity and inflammation in the body) and an ulcer frequency of up to 57%. These ailments can display as secondary issues such as ongoing pain, allergies, immune disorders, dermatological issues, pancreatitis, colitis, diarrhea, and regurgitation etc. with the cause often undiagnosed. Many of these dogs are under constant veterinarian care for various ailments. These issues can be seen in purebred and mixed breed dogs.

Known treatments involve the use of pharmaceutical products such as steroids, immune suppressants, NSAIDS, antibiotics, and pain medication. Diagnostic testing may include x-rays, scoping of the GI tract and expensive vet recommended processed feeds. Many dogs have recurring issues for which the known treatments can cost thousands of dollars.

### SUMMARY OF THE INVENTION

The inventors understand that the diet and lifestyle of horses and dogs contribute to their digestive health issues.

Processed horse feeds often contain lower grade by-products (soy hulls, wheat middlings, for example) which are nutrient deficient. As a result, many producers of such products add synthetic forms of vitamins and minerals. In addition, binding agents are used, some artificial like lignasol, or sugars like molasses which contain sulfur dioxide. The inventors understand that a horse's digestive system may be stressed by such fast digesting processed feeds containing high levels of sugar and chemical additives.

The inventors believe that in horses these diet factors in combination with limited natural turnout, heavy training, and stressful showing and traveling schedules can lead to colic, ulcers, diarrhea and leaky gut syndrome and the associated behavioral issues. Regardless of what supplements or feeds are added to the horse's diet to try to alleviate the symptoms, they often have an underlying malabsorption issue or transit time change due to a damaged, inflamed gastrointestinal tract.

The inventors understand that the modern dog diet contributes to digestive tract issues. Processed feeds may contain lower grade protein and by-products which are nutrient deficient (avian carcass for example). By-products are the internal remains of the animal excluding meat but may include diseased tissues, organs and tumors. Many producers of such products add synthetic forms of vitamins and minerals. In addition, binding agents and fillers are used such as corn and wheat gluten, food dyes and propylene glycol. The inventors understand that the dog's digestive system may be stressed by such feeds. Even natural foods may not be formulated or correctly balanced nutritionally. Food intolerances, allergies and skin issues are believed to be due, in part, to poor quality and biologically inappropriate ingredients in many commercial feeds.

The inventors believe that these diet factors in combination with natural environmental stressors or genetic predisposition may lead to leaky gut syndrome and associated secondary issues in dogs. Regardless of what supplements or feeds are added to the dog's diet, they may suffer from malabsorption issues due to a damaged, inflamed, or compromised gastrointestinal tract.

Formulation embodiments of the present invention are understood to aid in preventing and/or limiting the recurrence of these digestive tract issues, comprising a natural whole food supplement that is understood to improve gastrointestinal tract health by increased gastric muscle activity, allowing the animal to digest properly and absorb nutrients.

In one aspect, the present invention provides a formulation for alleviating digestive tract issues in animals, the formulation including: oat isolate, being oat flour prepared from de-hulled whole oat grain; hemp flour or meal containing no more than about 10 ppm tetrahydrocannabinol (THC); dehydrated cabbage; and dehydrated carrot; wherein; the dehydrated cabbage and dehydrated carrot are each prepared using low temperature dehydration, being a dehydration process in which the temperature does not exceed about 115 F; the oat isolate, hemp flour or meal, dehydrated cabbage and dehydrated carrot each have a moisture content of no more than about 10%; and a wt % of: the oat isolate is about 25 wt % to about 60 wt %; the hemp flour or meal is about 20 wt % to about 35 wt %; the dehydrated cabbage is about 5 wt % to about 20 wt %; and the dehydrated carrot is about 10 wt % to about 25 wt %.

The wt % of: the oat isolate may be about 30 wt % to about 50 wt %; the hemp flour or meal may be about 24 wt % to about 31 wt %; the dehydrated cabbage may be about 10 wt % to about 15 wt %; and the dehydrated carrot may be about 15 wt % to about 21 wt %.

The formulation may be for horses; and the wt % of: the oat isolate may be about 45 wt % to about 55 wt %; the hemp flour or meal may be about 22 wt % to about 28 wt %; the dehydrated cabbage may be about 8 wt % to about 12 wt %; and the dehydrated carrot may be about 12 wt % to about 18 wt %.

In the formulation for horses, the wt % of: the oat isolate may be about 50 wt %; the hemp flour or meal may be about 25 wt %; the dehydrated cabbage may be about 10 wt %; and the dehydrated carrot may be about 15 wt %.

In the formulation for horses, a unit dosage of the formulation may be about 70 grams to about 90 grams per 450 kg of horse weight in a daily feed schedule comprising two unit dosages. The unit dosage may be about 81 grams. The daily feed schedule may include two feedings spaced apart in time, with each feeding comprising one unit dosage.

In the formulation for horses, the moisture content of each of the oat isolate, hemp flour or meal, dehydrated cabbage and dehydrated carrot may be no more than about 6%. In the formulation for horses, the hemp flour or meal may contain no more than about 2 ppm THC.

The formulation may be for dogs; the formulation may include food grade diatomaceous earth (DE); and the wt % of: the oat isolate may be about 25 wt % to about 35 wt %; the hemp flour or meal may be about 25 wt % to about 35 wt %; the dehydrated cabbage may be about 10 wt % to about 20 wt %; the dehydrated carrot may be about 15 wt % to about 25 wt %; and the DE may be about 1 wt % to about 3 wt %.

In the formulation for dogs, the wt % of: the oat isolate may be about 31 wt %; the hemp flour or meal may be about 31 wt %; the dehydrated cabbage may be about 15 wt %; the dehydrated carrot may be about 20 wt %; and the DE may be about 2 wt %.

In the formulation for dogs, a unit dosage of the formulation may be about 1.5 grams to about 2.5 grams per 23 kg of dog weight in a daily feed schedule comprising two unit dosages. In the formulation for dogs, the unit dosage may be about 1 gram. In the formulation for dogs, the daily feed schedule may include two feedings spaced apart in time, with each feeding comprising one unit dosage.

In the formulation for dogs, the moisture content of each of the oat isolate, hemp flour or meal, dehydrated cabbage and dehydrated carrot may be no more than about 6%. In the formulation for dogs, the hemp flour or meal may contain no more than about 2 ppm THC.

### SUMMARY OF THE DRAWINGS

FIG. 1 is a bar graph illustrating contractile forces of non-glandular tissue exposed to acetylcholine (10⁻⁸-10⁻³), in which “*” denotes significant difference between Control (Co) and GS Formula (GF).

FIG. 2 is a bar graph illustrating contractile forces of margo plicatus tissue exposed to acetylcholine (10⁻⁸-10⁻³).

### DESCRIPTION OF EMBODIMENTS

Embodiments of the present invention include a formulation for horses with the following ingredients: oat isolate (Beta Glucan); and cabbage, dehydrated, flakes or powder, dried. A preferred embodiment of the present invention is a formulation for horses with the following ingredients: oat isolate (Beta Glucan); cabbage, dehydrated, flakes or powder, dried; Hemp, meal or flour from seed, dried, less than 10 ppm THC; and carrot, dehydrated, flakes or powder.

Embodiments of the present invention include a formulation for dogs with the following ingredients: oat isolate (Beta Glucan); cabbage, dehydrated, flakes or powder, dried; Hemp, meal or flour from seed, dried, less than 10 ppm THC; carrot, dehydrated, flakes or powder, dried; and food grade diatomaceous earth (DE).

The inventors understand that a usable moisture content of all ingredients may be in the range of about 5% to 10%, but preferably the moisture content is no more than about 6%. Preferably, the producers follow Good Manufacturing Guidelines (GMP) to ensure the following results: Yeast and molds: maximum of 500 cfu/gram (colony forming units); Peroxides: less than 20 meq/kg fat (milliequivalents/kg of fat); Water Activity: less than 0.75; and Total Plate Count: Maximum of 100,000 cfu/g.

The ingredients are whole foods that have not been heat processed (enzymes are de-natured and destroyed above 115 F) and all ingredients are preferably graded for human consumption.

Oat Isolate—Oat flour (steel milled oat groats). Oats are a source of beta glucans, which are understood to maintain regular rate of food passage, and prevent undigested starches from getting into the hind gut. Hindgut has flora specific for fermentation of fiber. The presence of undigested sugar or starches here can cause rise of lactic acid producing bacteria leading to ulcers or other hindgut issues (acidosis). Beta glucans are also understood to activate initial immune responses, and aid in elimination of toxins. (The initial immune response involves the activation of killer cells which respond initially before T and B cells can fully mobilize).

The inventors understand that the processing of some commercially available oat flour significantly reduces beta glucans. It is preferable to use a whole grain with only the hull removed, which retains up to 70% of original nutrients, with the beta glucans retained in the endosperm.

The whole oat is de-hulled (there are numerous types of de-hulling machines) which removes the indigestible outer husk. The resulting oat groat is sent through a steel cutter which splits the groat into 3 or 4 pieces hence the name steel cut oats. These oats are then processed into flour using flour mills which can include small kitchen appliances up to commercial mills ie stone mills, steel burr mills, roller mills, hammer mills etc. These processes use limited heat and therefore do not alter the nature of the oat.

Hemp meal (steel milled after oil pressed out). Hemp meal is understood to be a good source of digestible proteins (up to 50% protein, and contains all 20 amino acids and all 9 essential amino acids), and a good source of omega 3, 6 and 9 in proper ratios. Hemp meal is understood to provide a readily digestible plant protein, thus reducing the need for cereal grains which may be detrimental to gut health. Hemp meal is understood to be free from estrogen mimickers, lactose, gluten and trypsin, which enables it to be easily absorbed.

The main hemp derived feed-related products are hemp seed and hemp seed oil. Hemp production is often regulated. In Canada, all commercial industrial hemp growers use pedigreed seeds listed by Health Canada and are strictly regulated. These seeds produce plants that have less than 0.3% THC in leaves and flowering parts and less than 10 ppm in any products derived from the hemp grain. The inventors understand that there is no THC in the actual seed, and that any THC found when seeds are tested is from small amounts of the plant attached to the seed after cleaning. Manufacturers use extruders to remove the oil from the seed leaving a product they call hemp cake. This cake is then further refined into hemp meal or flour using any number or mill types as mentioned above. The hemp meal and flour used in the embodiments of the present invention preferably tests at no more than 2 ppm THC.

Cabbage—Blend of red and green cabbage (dehydrated at low temp so enzymes are not destroyed). Cabbage is a source of L Glutamine (amino acid) which is understood to balance stomach acid. Cabbage is also a source of choline, a B vitamin and lipotropic nutrient, and assists in maintaining cell membrane structure. Cabbage is also a source of Folate, a vitamin that assists in tissue repair, and is of use in the regeneration of the digestive tract lining, as these cells are replaced as often as every 4 days. Cabbage is also a source of S-methylmethionine which is understood to promote ulcer healing.

Carrot (dehydrated at low temp so enzymes are not destroyed). Carrots are understood to be a good source of folic acid (B9), beta carotene, Vitamin A and antioxidants (phytochemicals).

Both carrot and cabbage are prepared in a similar manner. After cleaning and chopping they are dehydrated at low temperature, to reduce moisture content. Once dehydrated, they are flaked or powdered. The flaked or powdered product is tested for moisture.

Commercial dehydrators suitable for the desired low-temperature dehydration include: vacuum, infrared microwave, osmotic, thermal, NutraREV, radiant energy etc. If dehydration is obtained in a conventional oven, a temperature maximum of 115F, or approximately 45C, should not be exceeded and the heating should be maintained until a desired moisture content is achieved, preferably until the ingredient has a moisture content under 6%. The carrot and cabbage may also be sun-dried, by being set in a sunny location above 85F with low humidity and insect control.

Diatomaceous earth is single celled honeycomb structured algae organisms encased in a silicon exo-skeletons, consisting of 33% silica, 19% calcium, 5% sodium, 3% magnesium and 2% iron, and typically including up to about 15 other minerals such as copper and manganese. DE is available in two grades, industrial and food grade. To be considered food grade DE must test for no more than 10 mg/kg of arsenic and 10 mg/kg of lead. The present invention uses food grade DE.

The inventors understand that diatomaceous earth is a natural detoxifying agent in that it has an anti-parasite and anti-viral effect, and cleanses the digestive tract. DE is also understood to boost liver function and is beneficial in absorbing harmful toxins from the blood. The inventors understand that DE retains its traits as a stable particle even when suspended in liquid medium. It carries an electrical charge allowing it to act as an anti-oxidant, by attracting free radicals, neutralizing them and carrying them from the body. DE is also understood to support bone and connective tissue health.

In a preferred embodiment for horses (referred to at times herein as the “GS Formulation” or “GSF”), the ingredients in a scoop (1 scoop equals 81 grams) are as follows: 40 g oat flour; 20 g hemp flour; 12.5 g dehydrated carrot; and 8.5 g dehydrated cabbage.

The inventors understand that the following ranges of ingredients would be suitable for horses: 1 gram to 2.2 kg per day of oat flour; up to 1 kg per day of hemp flour or meal; up to 1 kg per day of dehydrated carrot; and 1 gram to 50 grams per day of dehydrated cabbage.

In a preferred embodiment for dogs, the dosage is 2 grams orally twice a day per 50 lb or 23 kg dog, with each dose containing: 0.63 grams oat flour; 0.63 grams hemp flour; 0.30 grams cabbage; 0.40 grams carrot; and 0.04 grams Diatomaceous earth. The inventors understand that the following ranges of ingredients would be suitable for dogs (dose per 50 lb or 23 kg dog): Oats: 0.1 gram to 5 grams; Hemp: 0.1 gram to 5 grams; Cabbage: 0.1 gram to 3 grams; Carrot: 0.1 gram to 5 grams; and DE: up to 1 gram.

The GS Formulation embodiment was found to alleviate digestive tract issues in a seven-month trial with over 40 horses, which indicated that a suitable feed schedule for the above preferred embodiment is 1 scoop (81 grams) orally twice daily per 450 kg horse. The feed schedule can be adjusted for horses of different weights and, depending on the apparent effectiveness in alleviating digestive tract issues, can be halved as a maintenance regimen, or increased to tolerance.

Experiments to explore the application of the GS Formula (GSF) to gastrointestinal dysfunction in horses indicate a sensitizing effect of GSF on contractile response of gastric smooth muscle to a spasmogenic agent [acetylcholine (ACH)], suggesting application of GSF to conditions associated with delayed gastrointestinal transit time, such as impaction colic and re-feeding of malnourished horses.

In summary, the experiments involved an in vitro model of gastric contractility which was used to determine direct effects of a simulated digestion of GSF on sensitivity to a ACH aimed at providing insight into the most appropriate application for equine gastric health. A desensitizing to ACH suggests the application of the compound being investigated to gastric ulceration, as increased gastric contractility is associated with development of gastric ulcers. Conversely, a sensitizing effect on gastric smooth muscle response to ACH may indicate a better application of the product to conditions associated with delayed gastrointestinal transit time, such as impaction colic and re-feeding of malnourished horses. The data from the experiments demonstrated a sensitizing effect of GSF on contractile response of gastric smooth muscle to ACH.

The experimental method involved simulated digestion. In a living animal, gastric smooth muscle is deep to gastric mucosa and therefore is never directly exposed to gastric contents. Thus, any biological activity (i.e., effect on gastric smooth muscle) of a dietary substance will most likely be due to post-absorptive, metabolic end products of the substance. For this reason, the experimenters produced an extract of GSF that mimics effects of upper GIT digestion, absorption across biological membranes, and first-pass metabolism by the liver (Pearson W, Fletcher R S, Kott L S, Hurtig M B. Protection against LPS-induced cartilage inflammation and degradation provided by a biological extract of Mentha spicata. BMC Complement Altern Med. 2010 May 11; 10:19.). The dose of GSF extract used in the experiments described below was calculated based upon a single 160 g dose of GSF distributed in 330 L of total body water of the average 500 kg horse (Forro et al. 2000 Forro M, Cieslar S, Ecker G L, Walzak A, Hahn J, Lindinger M I. Total body water and ECFV measured using bioelectrical impedance analysis and indicator dilution in horses. J Appl Physiol (1985). 2000 August; 89 (2):663-71).

Selection of muscle samples from tissue—A challenge with conducting in vitro gastric research in horses is that there is a high degree of variability between individuals with respect to the health of their gastric tissue. Tissue must be collected post-mortem, which requires selection of tissue from horses of widely differing age, breed, and nutritional and health history. And often the investigators have little information on the background of the horses, so controlling for these variables can be almost impossible. Thus, for the purpose of the current experiments the experimenters opted to use tissue from abattoir pigs. Pigs are a suitable model for horses because, unlike humans and many other monogastrics which have glandular mucosa covering virtually the entire stomach, both pigs and horses have composite stomachs (ie. glandular and non-glandular squamous regions, separated by a cardiac region). The experimenters used tissue from all 3 regions of 8 pigs, in order to determine the effect of GSF extract on contractility of the smooth muscle portion of each region.

For each region (non-glandular—n; margo plicatus—m, and glandular—g), the control (CO) and experimental (GFS) sections were cut from the same piece. The CO and GSF sections were subdivisions of a larger piece of gastric smooth muscle which was dissected from the stomach. A section was cut to approximately 0.5×0.5 cm and the mucosal surface was completely removed, leaving only the smooth muscle portion.

Silk sutures were used to tie each tissue sample to a force transducer (which measured contraction force of each piece of tissue), and the tissue was submerged in an organ bath containing 25 mL of Krebs-Henseleit solution (KHS), which mimics the composition of extracellular fluid. A total of 6 tissue baths were set up to accommodate all sections such that they could all be run in one experiment, which enabled the experimenters to test all regions from within the same pig at the same time. The order of the CO and GFS sections were alternated to account for any potential variability associated with the location of the organ bath.

ACH Titration—Contractility of smooth muscle in a living horse is induced primarily by the neurotransmitter acetylcholine (ACH). In the horse's body, ACH crosses the junction between nerves and muscle cells and causes the muscle to contract. This is achieved through coordinated interactions between many cell products, including electrolytes, nerve cells, calcium and proteins. While it is known that (depending on the muscle) very small amounts of ACH are required to stimulate muscle contraction in the live animal, the very complex physiological systems which regulate muscle contraction are incompletely represented in the isolated muscle preparation described above. Thus, the experimenters conducted a titration experiment with ACH to determine the dose of ACH required to significantly increase contractility on samples from each region, both in the presence (GSF) and absence (CO) of GSF extract. The experimenters began with a very low ACH concentration then increased the concentration by removing 2.5mL of KHS and replacing it with ACH solution of 10-fold higher concentration than the previous exposure dose. This resulted in a dose response range of 10⁻⁸-10⁻³ M.

Data analysis—Contractile force was calculated by subtracting the mean baseline force of the tissue from the mean force generated for one minute following stimulation with ACH. This was then divided by the wet weight of the tissue section to generate a mean force/g. Contractile responses to ACH in the presence or absence of GSF were compared using the GLIMMIX procedure in SAS 9.4 (SAS Institute Inc.).

Interim Results—The results reported here are from 8 pigs.

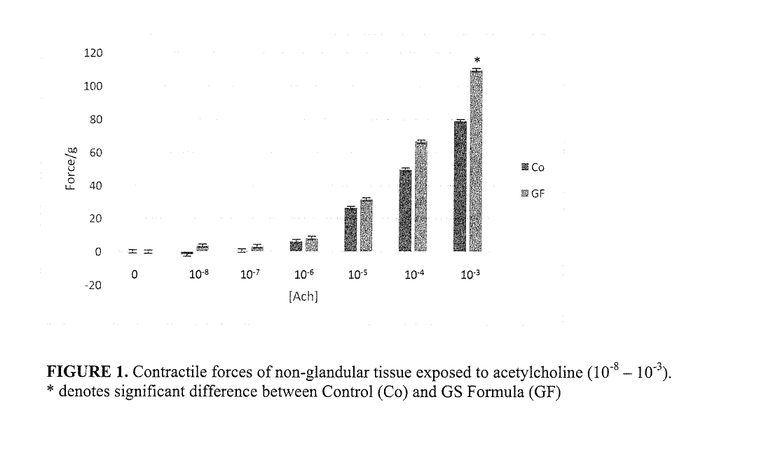

Non-glandular region—As illustrated in FIG. 1, ACH significantly increased contractility of muscle from the non-glandular region both in the presence and absence of GSF at ACH dose of 10-5 through 10-3. The contractile force was significantly higher in tissue conditioned with GSF at an ACH stimulus of 10-3 M (p=0.03).

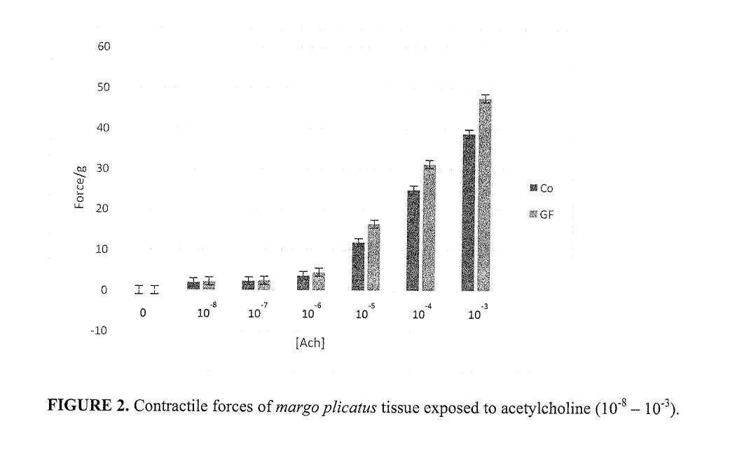

Margo Plicatus—As illustrated in FIG. 2, ACH significantly increased contractility of muscle from the margo plicatus region both in the presence and absence of GSF at ACH dose of 10-4 through 10-3 and there was no significant difference in contractility between Co and GSF tissue. However in GSF-treated tissue, ACH stimulation also produced significant contraction at a concentration of 10-5, which was not seen in the Co tissue.

Glandular region—Smooth muscle from this region of the stomach was very thick and was not well-suited to the experimental apparatus. Frequently when the tissue contracted it broke free of the silk ties and the experimenters were not able to measure forces.

What is claimed is:

1. A formulation for alleviating digestive tract issues in animals, the formulation comprising:
oat isolate, being oat flour prepared from de-hulled whole oat grain;
hemp flour or meal containing no more than about 10 ppm tetrahydrocannabinol (THC);
dehydrated cabbage; and
dehydrated carrot;
wherein;
the dehydrated cabbage and dehydrated carrot are each prepared using low temperature dehydration, being a dehydration process in which the temperature does not exceed about 115 F;
the oat isolate, hemp flour or meal, dehydrated cabbage and dehydrated carrot each have a moisture content of no more than about 10%; and
a wt % of:
the oat isolate is about 25 wt % to about 60 wt %;
the hemp flour or meal is about 20 wt % to about 35 wt %;
the dehydrated cabbage is about 5 wt % to about 20 wt %; and
the dehydrated carrot is about 10 wt % to about 25 wt %.

2. The formulation of claim 1, wherein the wt % of:
the oat isolate is about 30 wt % to about 50 wt %;
the hemp flour or meal is about 24 wt % to about 31 wt %;
the dehydrated cabbage is about 10 wt % to about 15 wt %; and
the dehydrated carrot is about 15 wt % to about 21 wt %.

3. The formulation of claim 1, wherein:
the formulation is for horses; and
the wt % of:
the oat isolate is about 45 wt % to about 55 wt %;
the hemp flour or meal is about 22 wt % to about 28 wt %;
the dehydrated cabbage is about 8 wt % to about 12 wt %; and
the dehydrated carrot is about 12 wt % to about 18 wt %.

4. The formulation of claim 3, wherein the wt % of:
the oat isolate is about 50 wt %;
the hemp flour or meal is about 25 wt %;
the dehydrated cabbage is about 10 wt %; and
the dehydrated carrot is about 15 wt %.

5. The formulation of claim 4, wherein a unit dosage of the formulation is about 70 grams to about 90 grams per 450 kg of horse weight in a daily feed schedule comprising two unit dosages.

6. The formulation of claim 5, wherein the unit dosage is about 81 grams.

7. The formulation of claim 5, wherein the daily feed schedule comprises two feedings spaced apart in time, with each feeding comprising one unit dosage.

8. The formulation of claim 3, wherein the moisture content of each of the oat isolate, hemp flour or meal, dehydrated cabbage and dehydrated carrot is no more than about 6%.

8. The formulation of claim 3, wherein the hemp flour or meal contains no more than about 2 ppm THC.

9. The formulation of claim 1, wherein:
the formulation is for dogs;
the formulation further comprises food grade diatomaceous earth (DE); and
the wt % of:
the oat isolate is about 25 wt % to about 35 wt %;
the hemp flour or meal is about 25 wt % to about 35 wt %;
the dehydrated cabbage is about 10 wt % to about 20 wt %;
the dehydrated carrot is about 15 wt % to about 25 wt %; and
the DE is about 1 wt % to about 3 wt %.

10. The formulation of claim 9, wherein the wt % of:
the oat isolate is about 31 wt %;
the hemp flour or meal is about 31 wt %;
the dehydrated cabbage is about 15 wt %;
the dehydrated carrot is about 20 wt %; and
the DE is about 2 wt %.

11. The formulation of claim 10, wherein a unit dosage of the formulation is about 1.5 grams to about 2.5 grams per 23 kg of dog weight in a daily feed schedule comprising two unit dosages.

12. The formulation of claim 11, wherein the unit dosage is about 1 gram.

13. The formulation of claim 11, wherein the daily feed schedule comprises two feedings spaced apart in time, with each feeding comprising one unit dosage.

14. The formulation of claim 9, wherein the moisture content of each of the oat isolate, hemp flour or meal, dehydrated cabbage and dehydrated carrot is no more than about 6%.

15. The formulation of claim 9, wherein the hemp flour or meal contains no more than about 2 ppm THC.
